(19) United States
(12) Patent Application Publication
(10) Pub. No.: US 2026/0192047 A1
(43) Pub. Date: Jul. 9, 2026

(54) MODEL-BASED COMPENSATION FOR ENHANCED INFUSION PUMP DELIVERY ACCURACY

(71) Applicant: CAREFUSION 303, INC., San Diego, CA (US)

(72) Inventors: Thomas John STANDLEY, San Diego, CA (US); Adonis Esahi GLASPER, San Diego, CA (US); Richard Stor WU, San Diego, CA (US); Alex William PINCHBECK, San Diego, CA (US)

(21) Appl. No.: 19/134,551

(22) PCT Filed: Dec. 1, 2022
(86) PCT No.: PCT/US2022/051576

§ 371 Date: May 30, 2025

(51) Int. Cl. A61M 5/168

(52) U.S. Cl. CPC A61M 5/16831; A61M 5/16877; A61M 2205/103; A61M 2205/3327; A61M 2205/3334; A61M 2205/3365; A61M 2205/3368; A61M 2205/502

(57) ABSTRACT

An infusion device includes a display and a pump configured for a predetermined volume per mechanical revolution (“VPMR”) under default environmental conditions. The infusion device receives infusion parameters that include (1) a requested flow rate of a fluid to be infused by the infusion device and, and (2) a head height and/or a patient height. The infusion device determines deviated environmental condition values as a function of (1) the received infusion parameters and (2) the default environmental conditions. The infusion device also determines a flow rate accuracy metric as a function of (1) the deviated environmental condition values and (2) an expected performance metric under the default environmental conditions. Responsive to determining the flow rate accuracy metric, the infusion device (a) displays, via the display, an indication of the flow rate accuracy metric; or (b) adjusts an operating speed of the pump based on the flow rate accuracy metric.

### BACKGROUND

During infusion therapies, every effort is made to maintain the flow of a medication to the patient at a particular flow rate. Assuming a standard clinical scenario, ensuring a given flow rate is sometimes as simple as translating the requested flow rate into an infusion pump operating speed. This translation is often performed based on a known variable referred to herein as volume per mechanical revolution (“VPMR”)—the amount of fluid that flows through the infusion pump per mechanical revolution of the pumping mechanism.

However, operating an infusion pump at a given flow rate is not always so simple. Factors external to the infusion pump can affect its VPMR, thus introducing inaccuracies into operating speed calculations. As inaccuracies creep into the calculations, risks of over- or under-reporting infused fluid volumes can arise which may impact, among other things, patient treatment. Accordingly, there is a need for an infusion pump that reduces flow rate inaccuracies by adjusting for external factors.

### SUMMARY

As noted, there is a need for an infusion pump that accounts for external factors in determining an ideal operating speed. Factors external to the infusion pump can affect its volume per mechanical revolution (“VPMR”) and flow rate accuracy, thus raising the risk of over- or under-infusion. For example, the vertical distance between the infusion pump and the patient, as well as the vertical distance between the infusion pump and the fluid container can each affect the infusion pump's VPMR and flow rate accuracy. Likewise, the type and temperature of the infusion fluid can impact the VPMR and the flow rate accuracy of the infusion pump. Other factors, discussed herein, can also affect the VPMR of the infusion pump. Accordingly, an infusion device adapted with features of the subject technology can receive or automatically detect external factors that may affect its VPMR and flow rate accuracy, and adjust its motor speed (or other operational characteristic) to account for said factors in order to better achieve a requested flow rate.

According to various aspects of the subject technology, an infusion device includes a display, a pump configured for a predetermined VPMR under default environmental conditions, a processor, and a non-transitory, computer-readable storage medium. The non-transitory, computer-readable storage medium stores instructions that, when executed by the processor, cause the infusion device to receive infusion parameters including (1) a requested flow rate of a fluid to be infused by the infusion device, and (2) a vertical distance between a fluid container and the infusion device, and/or a vertical distance between a patient and the infusion device. The instructions also cause the infusion device to determine deviated environmental condition values as a function of (1) the received infusion parameters and (2) the default environmental conditions. Additionally, the instructions cause the infusion device to determine a flow rate accuracy metric as a function of (1) the deviated environmental condition values and (2) an expected performance metric under the default environmental conditions. Further, the instructions cause the infusion device to, responsive to determining the flow rate accuracy metric: display, via the display, an indication of the flow rate accuracy metric; or adjust an operating speed of the pump based on the flow rate accuracy metric.

According to various aspects of the subject technology, a computer-implemented method for enhancing the accuracy of an infusion pump includes activating an infusion device that includes a display and a pump configured for a predetermined VPMR under default environmental conditions. The method also includes receiving infusion parameters including (1) a requested flow rate of a fluid to be infused by the infusion device, and (2) a vertical distance between a fluid container and the infusion device, and/or a vertical distance between a patient and the infusion device. Additionally, the method includes determining deviated environmental condition values as a function of (1) the received infusion parameters and (2) the default environmental conditions. Further, the method includes determining a flow rate accuracy metric as a function of (1) the deviated environmental condition values and (2) an expected performance metric under the default environmental conditions. Moreover, the method includes, responsive to determining the flow rate accuracy metric: displaying, via the display, an indication of the flow rate accuracy metric; or adjusting an operating speed of the pump based on the flow rate accuracy metric.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the Figures. Like reference numerals refer to corresponding parts throughout the Figures and Description.

FIG. 1 depicts an example institutional patient care system of a healthcare organization, according to aspects of the subject technology.

FIG. 2 illustrates an example head height and an example patient height in the context of an example infusion therapy, according to aspects of the subject technology.

FIG. 3 is an example table that includes seven example clinical scenarios, as well as respective infusion parameters and flow rate deviation metrics.

FIGS. 4A, 4B, and 4C are example user interfaces configured to provide and receive information regarding the accuracy of an infusion pump delivering an infusion, according to aspects of the subject technology.

FIG. 5 depicts an example line plot that illustrates the effect of flow rate on volume per mechanical revolution (“VPMR”), according to aspects of the subject technology.

FIG. 6 depicts an example process for improving the accuracy of an infusion pump, according to aspects of the subject technology.

FIG. 7 is a conceptual diagram illustrating an example electronic system for improving the accuracy of an infusion pump, according to aspects of the subject technology.

### DETAILED DESCRIPTION

Volume per mechanical revolution (“VPMR”) refers to the amount of fluid that flows through an infusion pump per mechanical revolution of the pump's pumping mechanism, for example, the amount of fluid that flows per camshaft revolution. Changes to environmental conditions may influence VPMR and thus cause variances in the pump's overall flow rate. The present disclosure provides a mechanism for correcting these flow rate variances based on identifying the changing environmental conditions and inputting the identified changes into a model.

According to various implementations, an infusion device is configured to receive infusion parameters, such as (1) a requested flow rate of a fluid to be infused by the infusion device, and (2) a vertical distance between a fluid container and the infusion device (e.g., a “head height”), and/or a vertical distance between a patient and the infusion device (e.g., a “patent height”). The infusion device then determines deviated environmental condition values as a function of (1) the received infusion parameters and (2) the default environmental conditions, and determines a flow rate accuracy metric as a function of (1) the deviated environmental condition values and (2) an expected performance metric under the default environmental conditions. Responsive to determining the flow rate accuracy metric, the infusion device may adjust an operating speed of the pump based on the flow rate accuracy metric, and/or display, via the display, an indication of the flow rate accuracy metric.

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In some instances, well-known methods, procedures, components, circuits, and networks have not been described in detail so as not to unnecessarily obscure aspects of the implementations.

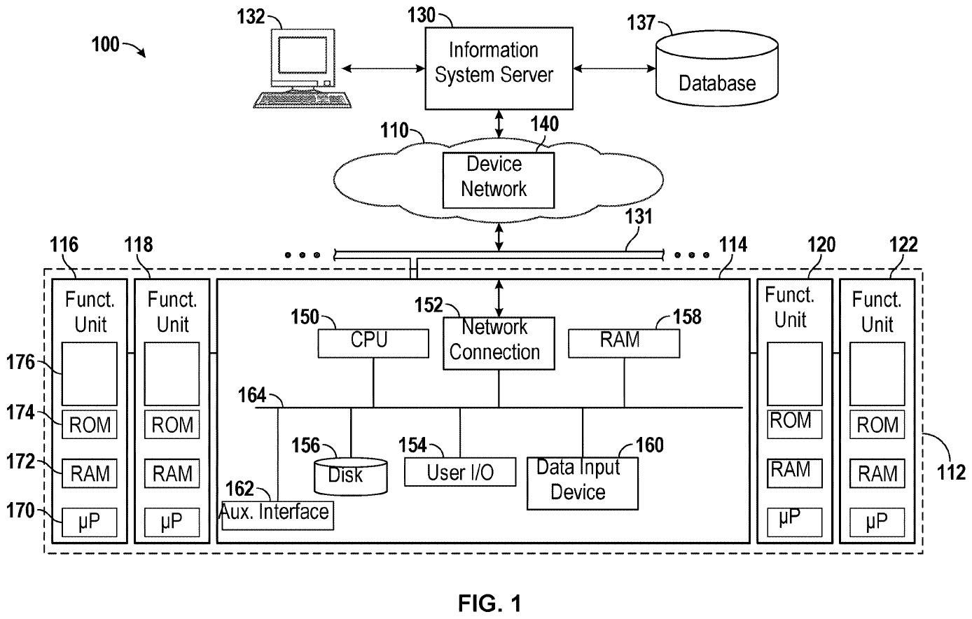

FIG. 1 depicts an example institutional patient care system 100 of a healthcare organization, according to aspects of the subject technology. In FIG. 1, a patient care device (“PCD”) 112 is connected to an internal healthcare network 110. The PCD 112 may include various ancillary medical devices such as an infusion pump, a vital-signs monitor, a medication dispensing device (e.g., a cabinet, a tote), a medication preparation device, an automated dispensing device, a module coupled with one of the aforementioned devices (e.g., a syringe pump module coupled with an infusion pump), or other similar devices. Each element of the PCD 112 is connected to the internal healthcare network 110 by a transmission channel 131. The transmission channel 131 may be a wired or wireless transmission channel (e.g., an 802.11 wireless local area network (LAN)).

In some implementations, the internal healthcare network 110 also includes computer systems located in various departments throughout a hospital. For example, the internal healthcare network 110 may include computer systems associated with an admissions department, a billing department, a biomedical engineering department, a clinical laboratory, a central supply department, one or more unit station computers, or a medical decision support system. As described below, the internal healthcare network 110 may also include discrete subnetworks. In the depicted example, the internal healthcare network 110 includes a device network 140 by which the PCD 112 and other devices may communicate in accordance with normal operations.

Additionally, the institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. The institutional patient care system 100 may further include a device terminal(s) 132 for connecting and communicating with information system server 130. The device terminal(s) 132 may include personal computers, personal data assistants, or mobile devices (e.g., laptops, tablet computers, augmented reality devices, smartphones) configured with software for communicating with the information system server 130 via the internal healthcare internal healthcare network 110.

The PCD 112 comprises a system for providing patient care, such as that described in U.S. Pat. No. 5,713,856 to Eggers et al., which is incorporated herein by reference for that purpose. The PCD 112 may also include or incorporate pumps, physiological monitors (e.g., a heart rate monitor, a blood pressure monitor, an ECG, an EEG, a pulse oximeter), therapy devices, or other drug delivery devices according to the teachings set forth herein. In the depicted example, the PCD 112 comprises an interface unit 114, connected to one or more functional modules 116, 118, 120, and 122. The interface unit 114 includes a central processing unit (CPU) 150 connected to a memory 158 (e.g., random access memory (RAM)) and one or more interface devices such as user interface device 154, a coded data input device 160, a network connection 152, and an auxiliary interface 162 for communicating with additional modules or devices. Additionally, in some implementations, the interface unit 114 includes a main, non-volatile storage unit 156 (e.g., a hard disk drive, a non-volatile flash memory) for storing software and data. Further, in some implementations, the interface unit 114 includes one or more internal buses 164 for interconnecting the aforementioned elements.

In various implementations, the user interface device 154 includes a touch screen for displaying information to a user and allowing a user to input information by touching defined areas of the screen. Additionally, or alternatively, the user interface device 154 could include means (specifically configured with one or more features described) for displaying and inputting information, such as a monitor, a printer, a keyboard, softkeys, a mouse, a track ball and/or a light pen.

The data input device 160 may be a bar code reader capable of scanning and interpreting data printed in a coded format (e.g., a bar code format, a QR code format). Additionally, or in the alternative, the data input device 160 can be a specifically-configured device for entering coded data into a computer, such as a device(s) for reading a magnetic strip, radio-frequency identification (RFID) devices whereby digital data encoded in RFID tags or smart labels are captured by the reader 160 via radio waves, Personal Computer Memory Card International Association (PCMCIA) smart cards, radio frequency cards, memory sticks, CDs, DVDs, or other analog or digital storage media. Other examples of the data input device 160 include a voice activation or recognition device or a portable personal data assistant (PDA). Depending upon the types of interface devices used, the user interface device 154 and the data input device 160 may be the same device.

Although the data input device 160 is shown as disposed within the interface unit 114, it is recognized that the data input device 160 may be integral within a pharmacy system, or located externally and communicating with the pharmacy system through an RS-232 serial interface or other appropriate communication means.

The auxiliary interface 162 may be an RS-232 communications interface. However, other means for communicating with a peripheral device in the described environments, such as a printer, patient monitor, infusion pump or other medical device, may be used without departing from the subject technology. Additionally, the data input device 160 may be a separate functional module, such as the functional modules 116, 118, 120 and 122. Further, the data input device 160 may be configured to communicate with the interface unit 114 or other systems on the internal healthcare network 110, using suitable programming and communication protocols.

The network connection 152 may be a wired or wireless connection, such as by Ethernet, WiFi, BLUETOOTH, an integrated services digital network (ISDN) connection, a digital subscriber line (DSL) modem, or a cable modem. Direct or indirect network connection may be used, including, but not limited to a telephone modem, an MIB system, an RS-232 interface, an auxiliary interface, an optical link, an infrared link, a radio frequency link, a microwave link, a personal area network (PAN) connection, a LAN connection, a cellular link, or a WLANS connection or other wireless connection.

The functional modules 116, 118, 120, and 122 are specially configured devices for providing care to a patient or for monitoring the condition of a patient. At least one of the functional modules 116, 118, 120, and 122 may be an infusion pump module such as an intravenous (IV) infusion pump for delivering medication or other fluid to a patient. For the purposes of this discussion, functional module 116 is an infusion pump module.

Each of functional modules 118, 120, and 122 may be a patient treatment or monitoring device including, but not limited to, an infusion pump, a syringe pump, a patient-controlled analgesia (PCA) pump, an epidural pump, an enteral pump, a blood pressure monitor, a pulse oximeter, an EKG monitor, an EEG monitor, a heart rate monitor, an intracranial pressure monitor, or the like. Functional modules 118, 120, and/or 122 may also be a printer, a scanner, a bar code reader, or another peripheral input, output, or input-output device.

Each of the functional modules 116, 118, 120, and 122 communicates directly or indirectly with the interface unit 114, with the interface unit 114 providing overall monitoring and control of the PCD 112. The functional modules 116, 118, 120, and 122 may be connected physically and electronically in serial fashion to one or both ends of interface unit 114 as shown in FIG. 1, or as detailed in Eggers et al. However, it is recognized that there are other means for connecting functional modules with the interface unit 114 that may be utilized without departing from the subject technology. It will also be appreciated that devices such as pumps or patient monitoring devices that provide sufficient programmability and connectivity may be capable of operating as stand-alone devices and may communicate directly with the network without being connected through a separate interface or control unit. As described above, additional medical devices or peripheral devices may be connected to the PCD 112 through the auxiliary interface 162. Each of the functional modules 116, 118, 120, and 122 may also include module-specific components 176, a microprocessor 170, a volatile memory 172, and a nonvolatile memory 174 for storing information.

It should be noted that, while four functional modules are shown in FIG. 1, any number of devices may be connected directly or indirectly to the interface unit 114. The number and type of functional modules described herein are intended to be illustrative, and they in no way limit the scope of the subject technology. The module-specific components 176 include specifically-configured components necessary for operation of a particular module, such as a pumping mechanism (e.g., a peristaltic pumping mechanism) for the infusion pump module 116.

While each functional module may be capable of a least some level of independent operation, the interface unit 114 monitors and controls overall operation of the PCD 112. For example, as will be described in more detail below, the interface unit 114 provides programming instructions to the functional modules 116, 118, 120, and 122 and monitors the status of each module.

The PCD 112 is capable of operating in several different modes (or “personalities”), with each mode defined by a configuration database. The configuration database may be a database 156 internal to the PCD 112, or it may be an external database 137. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics, or psychological characteristics.

As used herein, patient-specific information also includes care provider information (e.g., a physician identification) or the location of the PCD 112 in a hospital or the internal healthcare network 110. Patient care information may be entered through the network connection 152, the user interface device 154, the data input device 160, or the auxiliary interface 162. Additionally, patient care information may originate from anywhere in the internal healthcare network 110, such as, for example, from the device terminal 132, a pharmacy server, an admissions server, a laboratory server, and the like.

Medical devices incorporating aspects of the subject technology may be equipped with a network interface module (NIM), allowing the medical device to participate as a node in a network. For purposes of clarity, the subject technology will be described as operating in an Ethernet network environment using the Internet Protocol (IP); however, it is understood that concepts of the subject technology are equally applicable in other network environments, and such environments are intended to be within the scope of the subject technology.

Data to and from the various data sources can be converted into network-compatible data with existing technology, and movement of information between a medical device and a network can be accomplished by a variety of means. For example, the PCD 112 and the internal healthcare network 110 may communicate via automated interaction, manual interaction, or a combination of the two. Automated interaction may be continuous or intermittent and may occur directly through the network connection 152 (as shown in FIG. 1), or through RS-232 links, MIB systems, RF links such as Bluetooth, IR links, PANS, LANS, WLANS, digital cable systems, telephone modems, or other wired or wireless communication means.

Manual interaction between the PCD 112 and the internal healthcare network 110 involves physically transferring, intermittently or periodically, data between the systems using, for example, the user interface device 154, the data input device 160, bar codes, computer disks, portable data assistants, memory cards, or other media for storing data. The communication means in various aspects is bidirectional with access to data from as many points of the distributed data sources as possible. Decision-making can occur at a variety of places within the internal healthcare network 110. For example, and not by way of limitation, decisions can be made in the information system server 130, decision support, a remote data server, hospital department or unit stations, or within the PCD 112 itself.

All direct communications with medical devices operating on a network in accordance with the subject technology may be performed through the information system server 130, known as the remote data server (RDS). In accordance with aspects of the subject technology, network interface modules incorporated into medical devices (e.g., infusion pumps, vital-sign measurement devices) ignore all network traffic that does not originate from an authenticated RDS. The primary responsibilities of the RDS of the subject technology are to track the location and status of all networked medical devices that have NIMs, and maintain open communication.

In some implementations, the functional modules 116, 118, 120, and 122 include plug-in ports for expansion. Accordingly, an external medication delivery module may be attached to the PCD 112 by coupling a connector through a plug-in port (e.g., an electrical terminal) so that the external medication delivery module may transmit and receive information to and from the interface unit 114. In some implementations, the added external medication delivery module may also receive power from the interface unit 114 through a plug-in port.

The interface unit 114 may include a main display, a memory, and a processor. Additionally, the interface unit 114 may be configured to display operational parameters and medication delivery status, as well as information associated with each of the functional modules 116, 118, 120, and 122. According to various implementations, module displays may also display physiological data (e.g., vital signs) associated with a patient.

A main display (e.g., user interface device 154) may be configured to display one or more user interfaces for the display of operational parameters or other data associated with one or more of the functional modules 116, 118, 120, and 122, or physiological properties associated with the patient. The main display may include multiple user interfaces, with each individual user interface graphically displaying information for a respective one of the functional modules 116, 118, 120, and 122, including information that may also be displayed on corresponding module displays. In some implementations, the interface unit 114 includes a communications module (e.g., an antenna) configured to communicate wirelessly with a controller or with a network.

When one of the functional modules 116, 118, 120, and 122 initiates an infusion of a medication to a patient, the interface unit 114 is configured to create and manage an infusion session within a memory of the control module (or related module). For the purpose of this disclosure, the infusion session includes state information of the PCD 112, the interface unit 114, and/or the functional modules 116, 118, 120, and 122, which is recorded and saved to memory during a particular period of time. The state information includes, but is not limited to, records of parameter values utilized by the PCD 112, the interface unit 114, and/or the functional modules 116, 118, 120, and 122 during the period of time, and/or records physiological data collected during the period of time. During the infusion, physiological data associated with the patient is recorded within the session. Operating parameter values, as well as modifications to the operating parameters of the PCD 112, its control module, and/or modules are also recorded in the session.

If a clinician is not already logged into the PCD 112, the clinician may scan their badge proximate to a sensor (e.g., user interface device 154, data input device 160) on the PCD 112. Accordingly, the PCD 112 may then attempt to authenticate the clinician by sending the clinician's scanned identification to the information system server 130. The clinician's badge may incorporate a radio frequency identification device (RFID), which is read by a scanner integrated with the PCD 112 or a portable scanner associated with the PCD 112.

The clinician may scan their badge at the interface unit 114 to identify and authorize the clinician to initiate the administration of a medication. Once the clinician is associated with the PCD 112 and/or the functional modules 116, 118, 120, and 122, the clinician's identification is associated with the session. The same may also be applicable for a patient. For example, the clinician may scan a patient's wristband with a portable scanner or use the sensor on the PCD 112 (or that of the interface unit 114) to associate the patient with the PCD 112 and/or the functional modules 116, 118, 120, and 122 (and a session).

In some implementations, the interface unit 114 of PCD 112 is configured to generate a graphical representation of the infusion session, and display the graphical representation, including a graphical visualization of parameters of the infusion during the session and modifications to the parameters, together with physiological data obtained during the session. The graphical representation may include pseudo-identifiers for unknown data until such data is substituted with known identifiers. At that time, the graphical representation is displayed with the known patient identifiers.

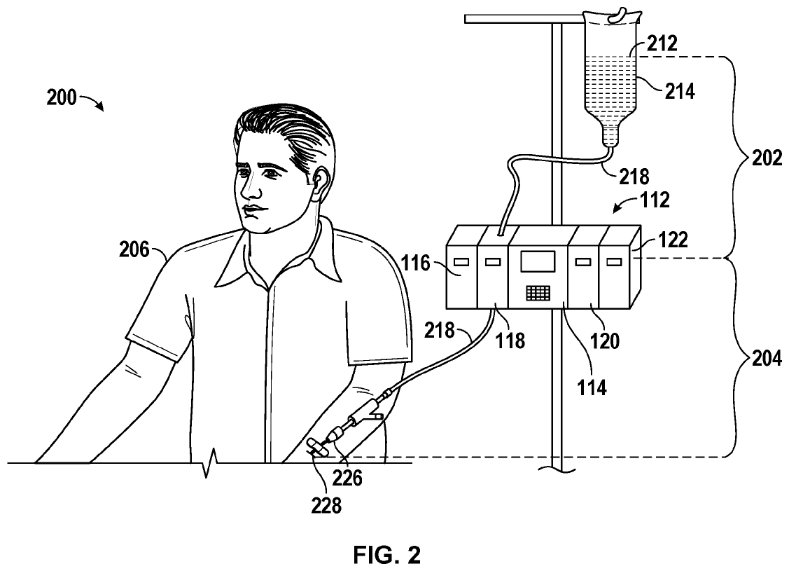

FIG. 2 illustrates an example head height 202 and an example patient height 204 in the context of an example infusion therapy 200, according to aspects of the subject technology. As depicted, the example infusion therapy 200 involves a patient 206 and the PCD 112. The PCD 112 includes the infusion pump module 116, which is configured to provide an IV fluid 212 (e.g., a medication, a saline solution) from an IV bag 214 to the patient 206. Additionally, the PCD 112 also includes the interface unit 114, as well as functional modules 118, 120, and 122.

The infusion pump module 116 is connected to the IV bag 214 via IV tubing 218. Additionally, the infusion pump module 116 is separated from the IV bag 214 by a vertical distance referred to as a head height 202. As used herein, “head height” refers to the vertical distance between an IV container (e.g., IV bag 214) and an infusion pump (e.g., infusion pump module 116). In some implementations, the head height 202 is measured as the vertical distance between the surface level of the IV fluid 212 in the IV bag 214 and the pumping mechanism of the infusion pump module 116.

Head height is one of many factors that may affect the VPMR and flow rate accuracy of an infusion pump. For example, if the head height 202 is increased (e.g., by raising the IV bag 214 and/or by lowering the infusion pump module 116), the VPMR of the infusion pump module 116 may increase as well (e.g., without a predetermined VPMR of the infusion pump reflecting the increase), which raises the risk of over-infusion. Likewise, if the head height 202 is decreased (e.g., by lowering the IV bag 214 and/or by raising the infusion pump module 116), the VPMR of the infusion pump module 116 may also decrease, risking under-infusion. In other words, if the head height 202 is different from a head height at which the infusion pump module 116 was calibrated, the flow rate accuracy of the infusion pump module 116 may be affected.

The infusion pump module 116 is also connected to the patient 206 at infusion site 228 via the IV tubing 218 and patient connector 226. Additionally, the infusion pump module 116 is separated from the patient 206 by a vertical distance referred to as a patient height 204. As used herein, “patient height” refers to the vertical distance between an infusion pump (e.g., infusion pump module 116) and a patient (e.g., patient 206). In some implementations, the patient height 204 is measured as the vertical distance between the pumping mechanism of the infusion pump module 116 and the infusion site 228 on the patient 206.

Like head height, patient height may also affect the VPMR of an infusion pump. For example, if the patient height 204 is decreased (e.g., by raising the infusion pump module 116 and/or by lowering the infusion site 228), the VPMR of the infusion pump module 116 may also increase (e.g., without a predetermined VPMR of the infusion pump reflecting the increase), which raises the risk of over-infusion. And, if the patient height 204 is increased (e.g., by lowering the infusion pump module 116 and/or by raising the infusion site 228), the VPMR of the infusion pump module 116 may decrease as well, risking under-infusion. If the patient height 204 is different from a patient height at which the infusion pump module 116 was calibrated, this may affect the accuracy of the infusion pump module 116.

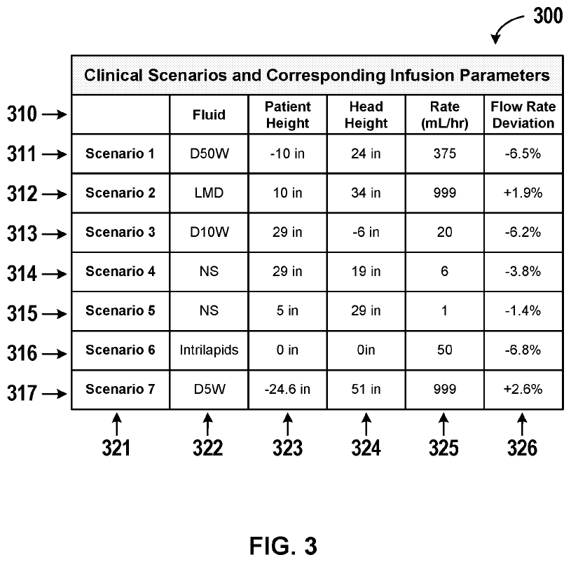

FIG. 3 is an example table 300 that includes seven example clinical scenarios (see rows 311-317), as well as respective infusion parameters (see columns 322-325) and flow rate deviation metrics (see column 326).

The table 300 is organized according to infusion parameters (indicated in a header row 310) and clinical scenarios (indicated in column 321). The infusion parameters identified in the header row 310 are, respectively, a fluid type (e.g., corresponding to a viscosity and/or a density of the fluid), a patient height, a head height, and a flow rate measured in milliliters per hour. Additionally, the table 300 includes a column 326 that identifies respective flow rate deviation metrics for each of the clinical scenarios.

Each clinical scenario 311-317 includes a different set of the aforementioned infusion parameters (see header row 310). For example, the first clinical scenario row 311 describes a clinical scenario that involves a dextrose 50% in water infusion (“D50 W”), at a patient height of −10 inches and a head height of 24 inches, and a flow rate of 375 milliliters per hour. Each clinical scenario also includes a respective flow rate deviation metric (see column 326). For example, the first clinical scenario 311 might result in a flow rate deviation of −6.5%. In other words, an infusion therapy performed with the infusion parameters identified in row 311 might under-infuse by 6.5% (e.g., resulting in an estimated flow rate of approximately 350 ml/hr).

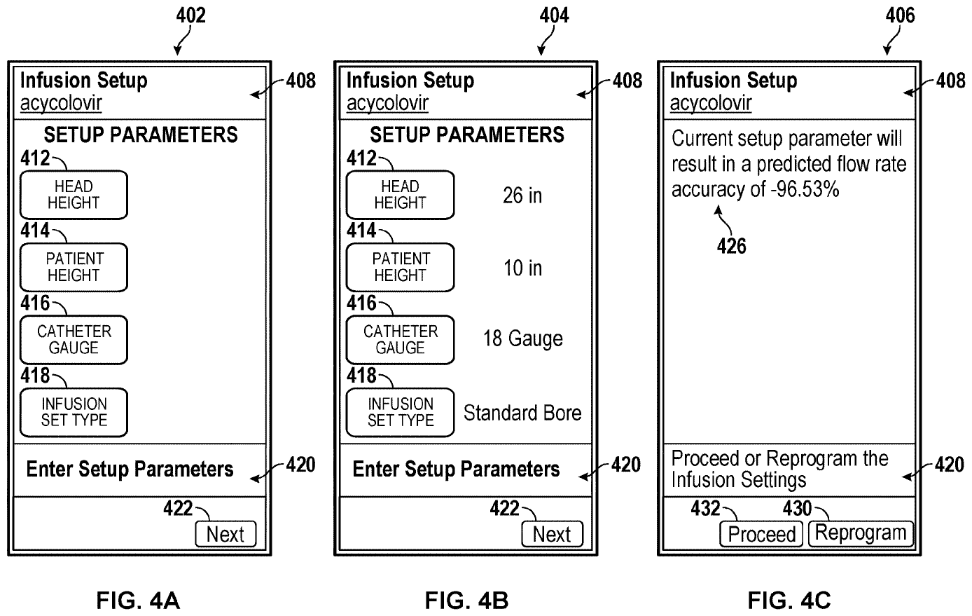

FIGS. 4A, 4B, and 4C are example user interfaces 402, 404, and 406 configured to provide and receive information regarding the accuracy of an infusion pump delivering an infusion, according to aspects of the subject technology. In some implementations, the user interfaces 402, 404, and 406 are displayed at an infusion control device (e.g., PCD 112), an infusion device (e.g., infusion module 116), or an external electronic device (e.g., device terminal 132).

The depicted example user interfaces 402, 404, and 406 each include a header 408 that indicates the device is currently in an “Infusion Setup” mode. In some implementations, the Infusion Setup mode includes displaying infusion parameter input fields 412, 414, 416, and 418 for an infusion therapy (e.g., infusion therapy 200). The input fields 412, 414, 416, and 418 may relate, for example, to infusion parameters that affect the accuracy of the infusion pump, such as a head height (e.g., head height 202), a patient height (e.g., patient height 204), a catheter gauge, or an infusion set type (e.g., of IV tubing 218). Other infusion parameter input fields may include a fluid type (e.g., of IV fluid 212), a filter type, or a requested flow rate.

In some implementations, the input fields 412, 414, 416, and 418 allow a user to input (e.g., via user interface device 154, data input device 160) infusion parameters. For example, a user may input a head height of 26 inches at input field 412, a patient height of 10 inches at input field 414, a catheter gauge of 18 at input field 416, and an infusion set type of “standard bore” at input field 418. Accordingly, the user interface 402 represents a user interface prior to receiving infusion parameters from a user. And the user interface 404 represents a user interface after receiving the aforenoted infusion parameters (e.g., a head height of 26 inches, etc.) from the user.

In some implementations, an input field (e.g., an additional input field) allows a user to input a fluid type (e.g., indicating a medication). The fluid type may be associated with a viscosity and/or a density. As with head height and patient height (and other infusion parameters, discussed herein), fluid viscosity and fluid density can also affect the VPMR and flow rate accuracy of the infusion pump. Accordingly, after receiving a fluid type (e.g., included in a request to infuse the fluid), the infusion device may lookup (e.g., in a database) the viscosity and/or density of the fluid to determine whether the VPMR or flow rate accuracy of the infusion pump will be affected.

According to various implementations, the user interfaces 402, 404, and 406 also include a prompt 420 indicating an action is required of the user (e.g., before user interface 404 will advance to user interface 406). For example, user interfaces 402 and 404 each include a prompt 420 that reads, “Enter setup parameters”. In some implementations, the prompt 420 of each of user interfaces 402 and 404 indicates that the user is required to enter infusion parameters before a next button 422 will function. For example, the next button 422 of the user interface 402 may be non-functional because the user has not yet entered the requested infusion parameters. On the other hand, the next button 422 of the user interface 404 may be functional because the user entered the setup parameters as requested in prompt 420.

The user interface 406 includes a prompt 420 that reads “Proceed or Reprogram the infusion settings”. In some implementations, the prompt 420 of the user interface 406 indicates that the user is required to either select a proceed button 432 or a reprogram button 430 in order to advance. The proceed button 432, for example, may initiate the infusion therapy without adjusting the operating speed of the infusion pump. The reprogram button 430, on the other hand, may initiate the infusion therapy after adjusting the operating speed of the infusion pump. Additionally, or in the alternative, the reprogram button 430 may recommend adjustments (e.g., adjusting a head height, adjusting a catheter gauge) to improve flow rate accuracy.

The user interface 406 may also include an indication 426 suggesting that the current infusion parameters (e.g., input via input fields 412, 414, 416, or 418 and/or automatically sensed by the infusion pump) will result in a predicted flow rate accuracy (i.e., a change in the predicted flow rate) of −3.47% (i.e., risking under-infusion). Accordingly, the user may determine that the predicted flow rate accuracy is acceptable and select the proceed button 432 to initiation the infusion therapy. Alternatively, the user may determine that the predicted flow rate accuracy is unacceptable and select the reprogram button 430. In some implementations, selecting the reprogram button 430 causes the infusion pump to adjust an operating speed of the infusion pump to achieve a requested flow rate.

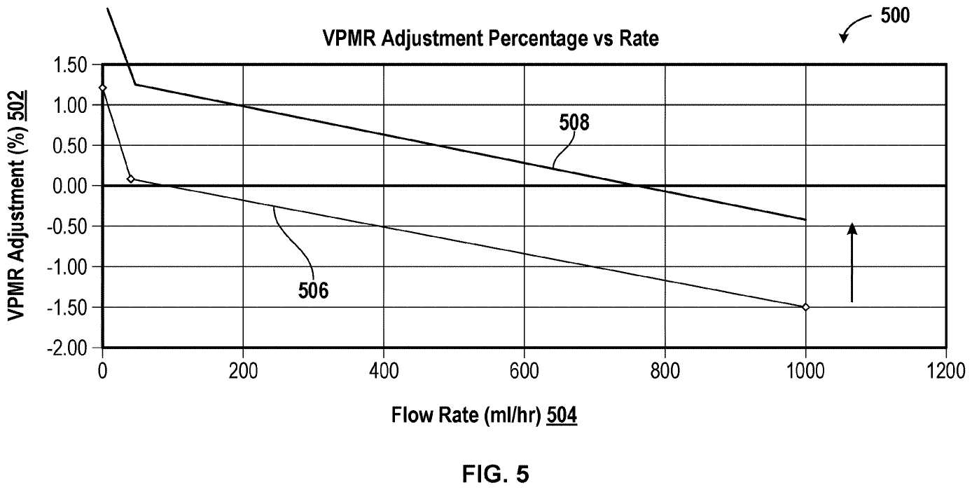

FIG. 5 depicts example calibration curves for correcting an infusion pump's volume per mechanical revolution (“VPMR”) under default conditions based on flow rate, according to aspects of the subject technology. As noted above, infusion rates may be translated into motor speeds using VPMR, where VPMR is the amount of fluid that flows through an infusion pump per mechanical revolution of the pump's pumping mechanism (e.g., the camshaft). Oftentimes, the configured VPMR of an infusion pump is approximately 180 μL.

VPMR varies slightly with infusion rate, and this variation may be taken into consideration in adjustments to the motor speed, for example, to correct for a pump's given VPMR (e.g., a calibrated VPMR). In this regard, each depicted curve 506, 508 may represent an amount of adjustment to the VPMR for setting the pump's performance to expected levels. The percentage adjustment (y-axis) may be the converse to the RPM of the motor. For example, an adjustment of −1.5% may impose an adjustment to the motor speed by 1.5%.

Each infusion pump's flow rate calculation may be based on a universal algorithm, which produces different results based on individualized parameter inputs. The depicted curves illustrate an example adjustment to a respective pump's VPMR (e.g., adjusting a programmed VPMR) to correct for variations from a given normalized value set (e.g, of environmental factors, such as head height, patient height, or fluid density) for the algorithm. The depicted first curve 506 may represent a default adjustment to VPMR based on flow rate, for example, according to manufacturer testing data. In some implementations, the expected performance of each pump may vary slightly under the same conditions for different flow rates. In this regard, the depicted second curve 508 may represent a second VPMR adjustment based on flow rate for a specific pump, or specific model of pumps.

According to the depicted example, when the infusion pump operates at a flow rate (e.g., a requested flow rate) of 90 ml/hr, the default VPMR adjustment percentage (e.g., for first curve 506) is approximately 0%—suggesting that the VPMR of the infusion pump is not expected to change when the infusion pump is operated at 90 ml/hr. In other words, if the infusion pump operates with a requested flow rate of 90 ml/hr, the actual flow rate of the infusion pump may also be 90 ml/hr. Likewise, if the infusion pump operates with a requested flow rate of 90 ml/hr, the actual VPMR of the infusion pump may be the same as the predetermined VPMR of the infusion pump (e.g., a VPMR for which the infusion pump was configured under default environmental conditions).

However, as another example, when the infusion pump operates with a flow rate (e.g., a requested flow rate) of 1000 ml/hr, the VPMR adjustment percentage may be −1.50%. Thus, the actual flow rate of an infusion pump operated with a requested flow rate of 1000 ml/hr may be less than the requested flow rate because the actual VPMR is 1.50% less than the predetermined VPMR. In this scenario, the infusion pump may under-infuse a medication or liquid. Similarly, the VPMR adjustment—and the resultant flow rate—at a flow rate approaching 0 ml/hr may be more than 1%. Accordingly, at exceptionally low flow rates, the infusion pump may over-infuse a medication or liquid.

Similarly, the second curve 508 represents an example adjustment (e.g., a linear adjustment) to the first curve 506 to account for the downward trend in VPMR adjustment as flow rate increases, for a respective pump or pump type/model. In the depicted example, the VPMR adjustment of the second curve 508 at a flow rate of 750 ml/hr is close to 0% (e.g., increasing flow rate accuracy). It is noted that that the second curve 508 depicted in the line plot 500 is offered by way of example and is not intended to limit the subject technology—the subject technology may, for example, include multiple different second curves (e.g., corresponding to different flow rates) or a curve with characteristics different than those depicted in line plot 500 (e.g., a curve that increases with flow rate).

As discussed in more detail below with respect to example process 600, the VPMR adjustment percentage may be obtained from a lookup table based on flow rate and, in some implementations, based on pump type and/or model to determine an additional constant a for determining a flow rate accuracy metric that may be applied to a calculation of the pump's motor speed, for example, to adjust the flow at which a medication is infused, and thereby causing the pump to delivery at a flow rate that is more accurately consistent with the pump's reported flow rate (e.g., based on internal calculations). This additional constant may be predetermined for correcting flow rate of a pump based on the dynamic VPMR adjustment (as shown in FIG. 5), stored in the lookup table, and obtained via the lookup table based on the flow rate.

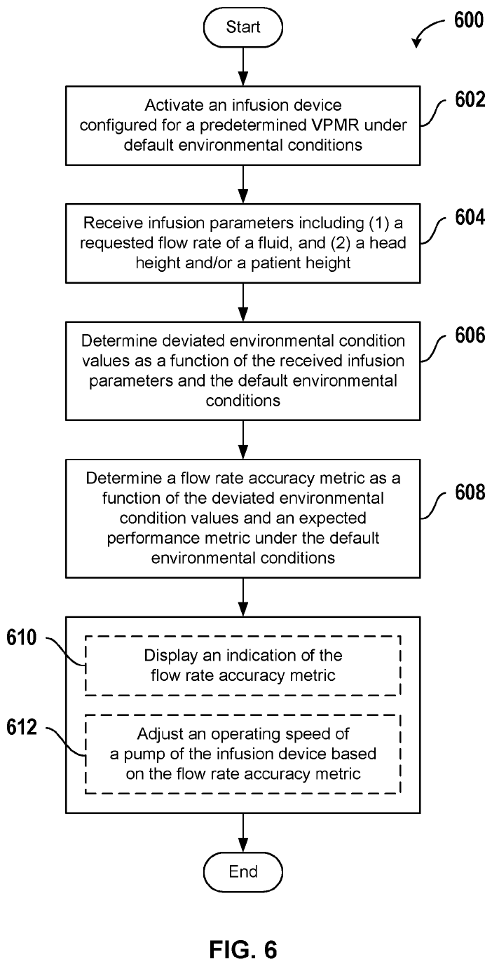

FIG. 6 depicts an example process 600 for improving the accuracy of an infusion pump, according to aspects of the subject technology. One or more blocks of process 600 may be implemented, for example, by one or more computing devices (e.g., PCD 112, infusion pump module 116, device terminal(s) 132). In some implementations, one or more of the blocks may be implemented based on one or more machine learning algorithms. In some implementations, one or more of the blocks may be implemented apart from other blocks, and by one or more different processors or devices. Further, for explanatory purposes, the blocks of example process 600 are described as occurring in serial, or linearly. However, multiple blocks of example process 600 may occur in parallel. Additionally, the blocks of example process 600 need not be performed in the order shown and one or more of the blocks of example process 600 need not be performed.

In the depicted example, the process 600 begins with activating an infusion device (e.g., PCD 112, infusion pump module 116) that is configured (e.g., at ROM 710) for a predetermined VPMR (602) under default environmental conditions. As noted previously, “VPMR” (volume per mechanical revolution) refers to an amount of fluid that is expected to flow through an infusion pump per mechanical revolution of the pumping mechanism of the infusion pump (e.g., per camshaft revolution). This value may vary dynamically based on flow rate, as described previously.

In this regard, the pump may be configured with a “predetermined VPMR” representative of a configured amount of fluid expected to flow through the infusion device per mechanical revolution of the under default environmental conditions (e.g., environmental conditions under which the infusion pump was calibrated). For example, the predetermined VPMR may be a static value (e.g., 0.180 mL) that does not account for changes in parameters external to the infusion pump (e.g., head height, patient height, fluid type).

In some implementations, the infusion device includes a display (e.g., user interface device 154). Additionally, the infusion device may also include one or more sensors, such as a temperature sensor, an upstream pressure sensor, a downstream pressure sensor, or an optical sensor. These sensors may be configured to detect a factor affecting the flow rate accuracy of the infusion pump (e.g., a fluid type, a patient height, a head height, an IV set type, a filter type, a catheter or needle type). Table 300, discussed above, provides several examples of how these factors may affect the delivery accuracy of an infusion device.

The depicted process 600 continues by receiving infusion parameters, such as a requested flow rate of a fluid to be infused by the infusion device, and a head height (i.e., the vertical distance between an IV container and an infusion pump) and/or a patient height (i.e., the vertical distance between an infusion pump and a patient) (604). In some implementations, the infusion parameters include a characteristic of the fluid to be infused by the infusion device, such as a density of the fluid, a viscosity of the fluid, or a temperature of the fluid.

In some implementations, the infusion parameters include a characteristic of an upstream tubing set that connects the infusion device to a fluid container, such as a length, an inner diameter, an elasticity, or an identifier of the upstream tubing set (e.g., an identifier that corresponds to another characteristic of the upstream tubing set). Additionally, in some implementations, the infusion parameters include a characteristic of a downstream tubing set that connects the infusion device to a patient (e.g., via a patient connector), such as a length, an inner diameter, an elasticity, or an identifier of the downstream tubing set (e.g., an identifier that corresponds to another characteristic of the downstream tubing set). Further, in some implementations, the infusion parameters include a characteristic of an accessory (e.g., a filter, a catheter, a needle, a patient connector), such as a length, an inner diameter, an elasticity, a flow resistance, or an identifier of the accessory (e.g., an identifier that corresponds to another characteristic of the accessory).

As described previously, the received infusion parameters may affect the VPMR of the infusion pump, thus impacting the flow rate of the pumping mechanism, and thus the overall accuracy of the pump with regard the flow rate which it reports. For example, when the patient height is positive (e.g., the infusion site 228 is higher than the infusion pump 112), the infusion pump may need to pump against gravity to deliver an IV fluid to a patient, and VPMR may decrease. On the other hand, if the patient height is negative (e.g., the infusion site 228 is lower than the infusion pump 112, as depicted in FIG. 2) gravity may assist delivery of the IV fluid with each mechanical revolution of the pumping mechanism (e.g., pumping cam), thereby increasing VPMR. According to various implementations, the received infusion parameters are used to adjust the flow rate of the pump to correct for changes to these environmental changes to VPMR.

In some implementations, the infusion parameters may be received via a user input. The user input may be obtained from a user, for example, via user interfaces 4A, 4B, or 4C. Additionally, or alternatively, the infusion parameters may be received via scanning a bar code or a QR code associated with the infusion therapy (e.g., a bar code of the IV bag 214, of the IV tubing 218). In some implementations, the infusion parameters may be automatically detected via the aforementioned sensors. For example, the infusion device may detect a temperature of the fluid via an inline temperature sensor. As another example, the infusion device may detect a patient height or a sensor height via an optical sensor or a pressure sensor (e.g., an upstream pressure sensor and/or a downstream pressure sensor).

Deviated environmental condition values are determined as a function of (1) the received infusion parameters and (2) default environmental conditions (606). In this regard, certain infusion parameters representative of characteristics of a fluid administration, such as dynamic pressure, upstream and downstream hydrostatic pressure, dwell time, and/or dynamic/elastic pressure, may be determined and adjusted based on certain infusion parameters representative of current environmental conditions, such as fluid type, temperature, patient height, head height, flow rate, and IV set type (e.g., tubing type). As will be described further, input parameters may input into a model to determine a flow rate accuracy metric which may be used to adjust the pump's flow rate (e.g., by adjusting the pump's motor speed) to correct for variations between the pump's actual flow rate and a desired flow rate.

In some implementations, the flow rate accuracy may be a metric that corresponds to a difference between the requested flow rate and an estimated flow rate (e.g., estimating the actual rate at which fluid will pass through the infusion pump). In some implementations, a flow rate accuracy metric of 100% denotes that the infusion pump is pumping accurately (e.g., the estimated VPMR matches the predetermined VPMR and/or the estimated flow rate matches the requested flow rate). In some implementations, a flow rate accuracy metric of 0% denotes that the infusion pump is pumping accurately (e.g., the actual flow rate deviates by 0% from the requested flow rate) (see, e.g., column 326 of table 300). In some implementations, the flow rate accuracy metric may be a value used to adjust a reported flow rate.

According to various implementations, a model, such as a machine learning model, an algorithm, and/or an equation, is used to generate the flow rate accuracy metric (608). The model receives the input parameters as inputs and determines the metric based on the input values. Input values may include, for example, infusion parameters and environmental condition values that may be obtained by way of user input and/or measurements by sensors associated with the pump during the infusion. In some implementations, the accuracy model may be based on the equation:

$$E = a + b * DDP + c * DHP + d * UHP + e * NPDT + f * UDEP \qquad ( 1 )$$

where E is the previously described flow rate accuracy metric. According to various implementations, E is represented as a percentage error with regard to the flow rate of the pump or a motor speed of the pump. Under optimal conditions, E may be zero (e.g., representing 0% error). For example, if the infusion parameters match the infusion parameters under which the predetermined VPMR was determined, the accuracy percentage error (E) may be 0% (e.g., because a, b, c, d, e, and f, are equal to 0). As another example, if the infusion pump is over-infusing (or expected to over-infuse) by three percent, the accuracy percentage error may be 3% (e.g., corresponding to a flow rate accuracy of 103%). In this regard, E may represent a difference between the actual and desired flow rate of the pump. Furthermore, E may be used to correct an increase or decrease in actual flow rate caused by current environmental conditions.

With regard to equation (1), DDP can represent the downstream dynamic pressure, which is measured in mmHg and may be a function of the requested flow rate, the fluid type (e.g., its viscosity), and the IV tubing (e.g., its length and inner diameter). Further, DHP can represent the downstream hydrostatic pressure, which may be measured in mmHg and may be a function of the fluid type (e.g., its density), and the patient height. Additionally, UHP can represent an upstream hydrostatic pressure, which may be measured in mmHg and may be a function of the fluid type (e.g., its density) and the head height. Moreover, NPDT can represent a negative pressure dwell time, which may be measured in seconds and may be a function of the fluid type (e.g., its density) and the patient height. UDEP can represent an upstream dynamic/elastic pressure, which may be measured in mmHg and may be a function of the IV tubing (e.g., its length, inner diameter), the requested flow rate, and the fluid type (e.g., the viscosity and/or density of the fluid).

According to various implementations, input variable a of the above equation (1) includes an expected performance metric under default environmental conditions. As explained herein, an infusion pump may be configured for a predetermined VPMR under default environmental conditions (e.g., a default head height, a default fluid temperature). With brief reference to FIG. 5, manufacturing testing may reveal small VPMR variations due to flow rate changes which, with minor correction, may be adjusted to obtain a more accurate flow rate from the pump. In this regard, a may factor into equation (1) to account for the effect of flow rate on VPMR (e.g., under default environmental conditions). In other words, a may be the inherent rate accuracy based on a default VPMR, as adjusted by flow rate (see, e.g., curve 506). In some implementations, a includes a residual error that remains after attempting to correct for VPMR (e.g., curve 508). During operation, a may be determined, for example, by way of indexing a lookup table based on flow rate. Additionally, or in the alternative, a may be representative of a calibration of VPMR, specifically for the given infusion pump or pump type/model.

According to various implementations, b, c, d, e, and f represent modifiers derived based on a difference between a set of infusion parameters (e.g., head height, patient height, fluid temperature) representative of current environmental conditions and the set of infusion parameters representing default environmental conditions. For example, variable b may be based aon the difference between the temperature of the fluid to be infused by the infusion pump and the temperature of the fluid used in configuring or calibrating the infusion pump. In this regard, these variables may be applied to metrics based on infusion parameters (e.g., DDP, DHP, UHP, NPDT, UDEP, discussed above) to generate deviated environmental condition values.

The deviated environmental condition values and/or the variables may be identified using machine learning or other artificial intelligence trained using historic or experimental infusion information (e.g., pump, firmware version, catheter or needle, etc.). For example, a user may provide received infusion parameters to a machine learning model which returns deviated environmental condition values and/or variables to the user.

In some implementations, the deviated environmental condition values and/or the variables may be based on the settings or features of the pump (e.g., firmware version, catheter or needle, height information). Moreover, in some implementations, the example process 600 determines whether the flow rate accuracy metric satisfies a predetermined deviation threshold, which may include, for example, a tolerance for a difference between the predetermined VPMR and the estimated VPMR, a difference between the requested flow rate and an estimated flow rate, or a difference between a requested delivery amount and an estimated delivery amount. In some implementations, the deviation threshold is set based on characteristics of the IV fluid 212. For example, if the IV fluid 212 is a medicine with strict delivery requirements (e.g., volume per period of time), the deviation threshold may be set relatively low (e.g., at ±1%). However, if the IV fluid 212 is a fluid (e.g., a saline solution) with less-strict delivery requirements, the deviation threshold may be set higher than that of the medicine with the strict delivery requirements (e.g., at ±5%). Similarly, the deviation threshold may be based on characteristics of the patient (e.g., a disease state, a height, a weight, a BMI, and/or an age of the patient). If the flow rate accuracy metric does not satisfy the deviation threshold then the example process 600 may proceed with initiating the programmed infusion therapy.

After determining the flow rate accuracy metric or, in some implementations, when the flow rate accuracy metric satisfies the deviation threshold, the example process 600 may include displaying (e.g., via the display of the infusion pump) an indication of the determined flow rate accuracy metric (610), and/or adjusting an operating speed of the pump based on the flow rate accuracy metric (612).

In some implementations, displaying the indication includes displaying a difference between the requested flow rate and an estimated flow rate (e.g., based on a difference between the predetermined VPMR and the estimated VPMR). When the operating speed is adjusted, the displayed indication may indicate the adjusted amount. In some implementations, displaying the indication includes displaying a difference between a requested total delivery amount and an estimated total delivery amount (e.g., based on the difference between the predetermined VPMR and the estimated VPMR). In some implementations, displaying the indication includes displaying a percentage difference or a unit difference (e.g., in mL for VPMR, mL/hr for flow rate, or mL for delivery amount). Further, in some implementations, displaying the indication includes displaying the flow rate accuracy metric itself (e.g., as a percentage). Additionally, or in the alternative, displaying the indication (610) may include displaying a graphical representation of the flow rate accuracy metric. For example, if the flow rate accuracy metric indicates a positive deviation (e.g., +5%, 105%), the indication may include a red, upward-pointing arrow or triangle.

When the flow rate accuracy metric indicates a deviation of +7% (i.e., risking over-infusion), adjusting the operating speed may include decreasing the operating speed of the pump to achieve the requested flow rate (e.g., decreasing the operating speed of the pump by 7%). Alternatively, if the flow rate accuracy indicates a deviation of −4% (i.e., risking under-infusion), adjusting the operating speed may include increasing the pump operating speed to achieve the requested flow rate (e.g., increasing the operating speed by 4%). In this regard, the operating speed of the infusion pump is adjusted to improve the flow rate accuracy (e.g., reduce flow rate accuracy deviation to 0%).

In some implementations, the process 600 includes periodically or continuously adjusting the operating speed of the infusion pump based on the requested flow rate and input from the one or more sensors. For example, an inline temperature sensor may indicate a sudden rise in the temperature of the IV fluid while an infusion therapy is ongoing. Accordingly, the flow rate accuracy may be re-determined in real time based on the new fluid temperature (and/or other infusion parameters), and the operating speed of the infusion pump may be adjusted accordingly. As another example, an optical sensor may indicate the position of the patient has changed, causing a change in the patient height. Accordingly, the flow rate accuracy may be re-determined based on the new patient height (and/or other infusion parameters), and the operating speed of the infusion pump may then be readjusted.

The process 600 may also include determining an actual flow rate of the infusion device, for example, via a flow sensor. The actual flow rate may then be used to further adjust (see operation 516) the operating speed of the infusion pump or to further train a model (e.g., a machine learning model, an equation, and/or an algorithm), as discussed above.

The process 600 may also include recommending that the user change the infusion parameters to achieve the requested flow rate. As noted above, the pump's VPMR may be calibrated based on a set of default infusion parameters, such as a default head height, a default patient height, a default IV set configuration, and so on. Accordingly, changing an infusion parameter such that it is closer to or matches a default infusion parameter may improve the flow rate accuracy. In this manner, the infusion pump may recommend that a user of the infusion pump adjust one or more infusion parameters (e.g., adjust the head height by adjusting the height of the IV container 214) such that they match or are at least closer to the corresponding default infusion parameters, in order to improve the flow rate accuracy (e.g., as an alternative to adjusting the operating speed of the infusion pump).

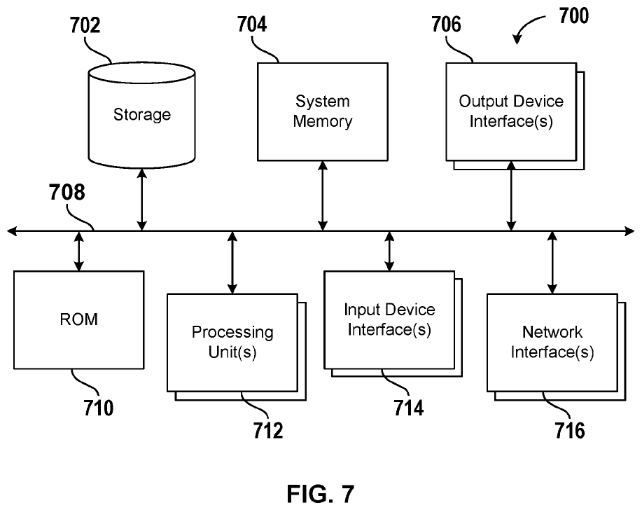

FIG. 7 is a conceptual diagram illustrating an example electronic system 700 for improving the accuracy of an infusion pump, according to aspects of the subject technology. The electronic system 700 may be implemented by a computing device for execution of software associated with portions or steps of the process 600, or components and methods provided by FIGS. 1-5. In this regard, the electronic system 700 may include the PCD 112 or the infusion pump module 116. The electronic system 700 may also include a specifically configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

The electronic system 700 may also include various types of computer readable media and interfaces for various other types of computer readable media. In the depicted example, the electronic system 700 includes a bus 708, a processing unit(s) 712, a system memory 704, a read-only memory (ROM) 710, a permanent storage device 702, an input device interface(s) 714, an output device interface(s) 706, and a network interface(s) 716. In some implementations, the electronic system 700 may include or be integrated with other computing devices or circuitry for operation of the various components and methods previously described.

The bus 708 collectively represents all system, peripheral, and chipset buses that communicatively connect the numerous internal devices of the electronic system 700. For instance, the bus 708 communicatively connects the processing unit(s) 712 with the ROM 710, the system memory 704, and the permanent storage device 702.

From these various memory units, processing unit(s) 712 retrieves instructions to execute and data to process, in order to execute the processes of the subject disclosure. The processing unit(s) 712 can be a single processor or a multi-core processor in different implementations.

The ROM 710 stores static data and instructions that are needed by the processing unit(s) 712 and other modules of the electronic system. The permanent storage device 702, on the other hand, is a read-and-write memory device. This device is a non-volatile memory unit that stores instructions and data even when the electronic system 700 is powered off. Some implementations of the subject disclosure use a mass-storage device (such as a magnetic or optical disk and its corresponding disk drive) as the permanent storage device 702. Other implementations use a removable storage device (such as a floppy disk, flash drive, and its corresponding disk drive) as the permanent storage device 702.

Like the permanent storage device 702, the system memory 704 is a read-and-write memory device. However, unlike the storage device 702, the system memory 704 is a volatile read-and-write memory, such as random-access memory (RAM). The system memory 704 stores some of the instructions and data that the processor needs at runtime. In some implementations, the processes of the subject disclosure are stored in the system memory 704, the permanent storage device 702, and/or the ROM 710. From these various memory units, the processing unit(s) 712 retrieves instructions to execute and data to process, in order to execute the processes of some implementations.

The bus 708 also connects to the input device interface(s) 714 and the output device interface(s) 706. The input device interface(s) 714 enables the user to communicate information and select commands to the electronic system. Input devices used with the input device interface(s) 714 include, for example, alphanumeric keyboards and pointing devices (also called “cursor control devices”). The output device interface(s) 706 enables, for example, the display of images generated by the electronic system 700. Output devices used with the output device interface(s) 706 include, for example, printers and display devices, such as cathode ray tubes (CRT) or liquid crystal displays (LCD). Some implementations include devices (e.g., touchscreens) that function as both input and output devices.

Furthermore, the bus 708 also couples the electronic system 700 to a network (not shown) through the network interface(s) 716. The network interface(s) 716 may include, for example, a wireless access point (e.g., Bluetooth or Wi-Fi) or radio circuitry for connecting to a wireless access point. The network interface(s) 716 may also include hardware (e.g., ethernet hardware) for connecting the computer to a part of a network of computers such as a local area network (LAN), a wide area network (WAN), wireless LAN, an intranet, or a network of networks, such as the Internet. Any or all components of electronic system 700 can be used in conjunction with the subject disclosure when specifically configured with one of more of the features described.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,”, “server”, “processor”, and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An infusion device, comprising: a display; a pump configured for a predetermined volume per mechanical revolution (“VPMR”) under default environmental conditions; a processor; and a non-transitory, computer-readable storage medium storing instructions that, when executed by the processor, cause the infusion device to: receive infusion parameters comprising (1) a requested flow rate of a fluid to be infused by the infusion device, and (2) a vertical distance between a fluid container and the infusion device, and/or a vertical distance between a patient and the infusion device; determine deviated environmental condition values as a function of (1) the received infusion parameters and (2) the default environmental conditions; determine a flow rate accuracy metric as a function of (1) the deviated environmental condition values and (2) an expected performance metric under the default environmental conditions; and responsive to determining the flow rate accuracy metric: display, via the display, an indication of the flow rate accuracy metric; or adjust an operating speed of the pump based on the flow rate accuracy metric.
Clause 2. The infusion device of claim 1, wherein determining the flow rate accuracy metric as a function of (1) the deviated environmental condition values and (2) the expected performance metric under the default environmental conditions comprises: providing the infusion parameters to a machine learning model; and receiving the flow rate accuracy metric from the machine learning model.
Clause 3. The infusion device of any one of claims 1-2, wherein the expected performance metric is determined based on the requested flow rate.
Clause 4. The infusion device of any one of Clauses 1 through 3, wherein the non-transitory, computer-readable storage medium further stores instructions that, when executed by the processor, cause the infusion device to: responsive to determining the flow rate accuracy metric: display, via the display, the indication of the flow rate accuracy metric; display, via the display, an option to adjust the operating speed of the pump; and adjust, responsive to receiving a confirmation of the option to adjust the operating speed of the pump, the operating speed of the pump based on the flow rate accuracy metric.
Clause 5. The infusion device of any one of Clauses 1 through 4, wherein receiving the infusion parameters comprises automatically detecting the infusion parameters via one or more sensors.
Clause 6. The infusion device of Clause 5, wherein automatically detecting the infusion parameters via one or more sensors comprises: automatically detecting, via an upstream pressure sensor, the vertical distance between the fluid and the infusion device; or automatically detecting, via a downstream pressure sensor, the vertical distance between the patient and the infusion device.
Clause 7. The infusion device of any one of Clauses 5 through 6, wherein: the infusion parameters further comprise a temperature of the fluid; and automatically detecting the infusion parameters via one or more sensors comprises automatically detecting, via a temperature sensor, the temperature of the fluid.
Clause 8. The infusion device of any one of Clauses 1 through 7, wherein receiving the infusion parameters comprises receiving the infusion parameters from a user input device.
Clause 9. The infusion device of Clause 8, wherein: the infusion parameters further comprise a density of the fluid or a viscosity of the fluid; and receiving the infusion parameters from the user input device comprises: receiving a type of the fluid from the user input device; and determining, based on the type of the fluid, the density of the fluid or the viscosity of the fluid.
Clause 10. The infusion device of any one of Clauses 1 through 9, wherein displaying the indication of the flow rate accuracy metric comprises displaying a percentage corresponding to the flow rate accuracy metric.
Clause 11. A computer-implemented method for enhancing accuracy of an infusion pump, comprising: activating an infusion device comprising a display and a pump configured for a predetermined volume per mechanical revolution (“VPMR”) under default environmental conditions; receiving infusion parameters comprising (1) a requested flow rate of a fluid to be infused by the infusion device, and (2) a vertical distance between a fluid container and the infusion device, and/or a vertical distance between a patient and the infusion device; determining deviated environmental condition values as a function of (1) the received infusion parameters and (2) the default environmental conditions; determine a flow rate accuracy metric as a function of (1) the deviated environmental condition values and (2) an expected performance metric under the default environmental conditions; and responsive to determining the flow rate accuracy metric: displaying, via the display, an indication of the flow rate accuracy metric; or adjusting an operating speed of the pump based on the flow rate accuracy metric.
Clause 12. The computer-implemented method of Clause 11, wherein determining the flow rate accuracy metric as a function of (1) the deviated environmental condition values and (2) the expected performance metric under the default environmental conditions comprises: providing the infusion parameters to a machine learning model; and receiving the flow rate accuracy metric from the machine learning model.
Clause 13. The computer-implemented method of any one of Clauses 11 through 12, wherein the expected performance metric is determined based on the requested flow rate.
Clause 14. The computer-implemented method of any one of Clauses 11 through 13, further comprising: responsive to determining the flow rate accuracy metric: displaying, via the display, the indication of the flow rate accuracy metric; displaying, via the display, an option to adjust the operating speed of the pump; and adjusting, responsive to receiving a confirmation of the option to adjust the operating speed of the pump, the operating speed of the pump based on the flow rate accuracy metric.
Clause 15. The computer-implemented method of any one of Clauses 11 through 14, wherein receiving the infusion parameters comprises automatically detecting the infusion parameters via one or more sensors.
Clause 16. The computer-implemented method of Clause 15, wherein automatically detecting the infusion parameters via one or more sensors comprises: automatically detecting, via an upstream pressure sensor, the vertical distance between the fluid and the infusion device; or automatically detecting, via a downstream pressure sensor, the vertical distance between the patient and the infusion device.
Clause 17. The computer-implemented method of any one of Clauses 15 through 16, wherein: the infusion parameters further comprise a temperature of the fluid; and automatically detecting the infusion parameters via one or more sensors comprises automatically detecting, via a temperature sensor, the temperature of the fluid.
Clause 18. The computer-implemented method of any one of Clauses 11 through 17, wherein receiving the infusion parameters comprises receiving the infusion parameters from a user input device.
Clause 19. The computer-implemented method of Clause 18, wherein: the infusion parameters further comprise a density of the fluid or a viscosity of the fluid; and receiving the infusion parameters from the user input device comprises: receiving a type of the fluid from the user input device; and determining, based on the type of the fluid, the density of the fluid or the viscosity of the fluid.
Clause 20. The computer-implemented method of any one of Clauses 11 through 19, wherein displaying the indication of the flow rate accuracy metric comprises displaying a percentage corresponding to the flow rate accuracy metric.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein but are to be accorded the full scope consistent with the language of the claims. For example, reference to an element in the singular is not intended to mean “one and only one” unless specifically so stated, but rather “one or more”. Moreover, unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic”, as used herein, may include performance by a computer or machine without user intervention, for example, by instructions responsive to a predicate action by the computer or machine or other initiation mechanism. The word “example” is used herein to mean “serving as an example or illustration”. Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface, or a UI) may refer to a network-based interface including data fields or other control elements for receiving input signals or providing electronic information or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fu5y logic, pattern matching, a machine learning assessment model, or combinations thereof.

As used herein, the language “[A] and/or [B]” (where [A] and [B] are placeholders for any element) is intended to cover situations in which the claimed feature includes [A], includes [B], or includes [A] and [B]. Similarly, the language “at least one of [A] or [B]” is intended to cover situations in which the claimed feature includes [A], includes [B], or includes [A] and [B].

In some implementations, data generated or detected can be forwarded to a “remote” device or location, where “remote” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

Aspects described include artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired item location. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features, which may be included in the embodiments discussed above, are described in “A survey of machine learning for big data processing” by Qiu et al. in EURASIP Journal on Advances in Signal Processing (2016) which is hereby incorporated by reference in its entirety.

1. An infusion device, comprising:
a display;
a pump configured for a predetermined volume per mechanical revolution (“VPMR”) under default environmental conditions;
a processor; and
a non-transitory, computer-readable storage medium storing instructions that, when executed by the processor, cause the infusion device to:
receive infusion parameters comprising (1) a requested flow rate of a fluid to be infused by the infusion device, and (2) a vertical distance between a fluid container and the infusion device, and/or a vertical distance between a patient and the infusion device;
determine deviated environmental condition values as a function of (1) the received infusion parameters and (2) the default environmental conditions; determine a flow rate accuracy metric as a function of (1) the deviated environmental condition values and (2) an expected performance metric under the default environmental conditions; and
responsive to determining the flow rate accuracy metric:
display, via the display, an indication of the flow rate accuracy metric; or
adjust an operating speed of the pump based on the flow rate accuracy metric.

2. The infusion device of claim 1, wherein determining the flow rate accuracy metric as a function of (1) the deviated environmental condition values and (2) the expected performance metric under the default environmental conditions comprises:
providing the infusion parameters to a machine learning model; and
receiving the flow rate accuracy metric from the machine learning model.

3. The infusion device of claim 1, wherein the expected performance metric is determined based on the requested flow rate.

4. The infusion device of claim 1, wherein the non-transitory, computer-readable storage medium further stores instructions that, when executed by the processor, cause the infusion device to:
responsive to determining the flow rate accuracy metric:
display, via the display, the indication of the flow rate accuracy metric;
display, via the display, an option to adjust the operating speed of the pump; and
adjust, responsive to receiving a confirmation of the option to adjust the operating speed of the pump, the operating speed of the pump based on the flow rate accuracy metric.

5. The infusion device of claim 1, wherein receiving the infusion parameters comprises automatically detecting the infusion parameters via one or more sensors.

6. The infusion device of claim 5, wherein automatically detecting the infusion parameters via one or more sensors comprises:
automatically detecting, via an upstream pressure sensor, the vertical distance between the fluid and the infusion device; or
automatically detecting, via a downstream pressure sensor, the vertical distance between the patient and the infusion device.

7. The infusion device of claim 5, wherein:
the infusion parameters further comprise a temperature of the fluid; and
automatically detecting the infusion parameters via one or more sensors comprises automatically detecting, via a temperature sensor, the temperature of the fluid.

8. The infusion device of claim 1, wherein receiving the infusion parameters comprises receiving the infusion parameters from a user input device.

9. The infusion device of claim 8, wherein:
the infusion parameters further comprise a density of the fluid or a viscosity of the fluid; and
receiving the infusion parameters from the user input device comprises:
receiving a type of the fluid from the user input device; and
determining, based on the type of the fluid, the density of the fluid or the viscosity of the fluid.

10. The infusion device of claim 1, wherein displaying the indication of the flow rate accuracy metric comprises displaying a percentage corresponding to the flow rate accuracy metric.

11. A computer-implemented method for enhancing accuracy of an infusion pump, comprising:
activating an infusion device comprising a display and a pump configured for a predetermined volume per mechanical revolution (“VPMR”) under default environmental conditions;
receiving infusion parameters comprising (1) a requested flow rate of a fluid to be infused by the infusion device, and (2) a vertical distance between a fluid container and the infusion device, and/or a vertical distance between a patient and the infusion device;
determining deviated environmental condition values as a function of (1) the received infusion parameters and (2) the default environmental conditions;
determining a flow rate accuracy metric as a function of (1) the deviated environmental condition values and (2) an expected performance metric under the default environmental conditions; and
responsive to determining the flow rate accuracy metric:
displaying, via the display, an indication of the flow rate accuracy metric; or
adjusting an operating speed of the pump based on the flow rate accuracy metric.

12. The computer-implemented method of claim 11, wherein determining the flow rate accuracy metric as a function of (1) the deviated environmental condition values and (2) the expected performance metric under the default environmental conditions comprises:
providing the infusion parameters to a machine learning model; and
receiving the flow rate accuracy metric from the machine learning model.

13. The computer-implemented method of claim 11, wherein the expected performance metric is determined based on the requested flow rate.

14. The computer-implemented method of claim 11, further comprising:
responsive to determining the flow rate accuracy metric:
displaying, via the display, the indication of the flow rate accuracy metric.
displaying, via the display, an option to adjust the operating speed of the pump; and
adjusting, responsive to receiving a confirmation of the option to adjust the operating speed of the pump, the operating speed of the pump based on the flow rate accuracy metric.

15. The computer-implemented method of claim 11, wherein receiving the infusion parameters comprises automatically detecting the infusion parameters via one or more sensors.

16. The computer-implemented method of claim 15, wherein automatically detecting the infusion parameters via one or more sensors comprises:
automatically detecting, via an upstream pressure sensor, the vertical distance between the fluid and the infusion device; or
automatically detecting, via a downstream pressure sensor, the vertical distance between the patient and the infusion device.

17. The computer-implemented method of claim 15, wherein:
the infusion parameters further comprise a temperature of the fluid; and
automatically detecting the infusion parameters via one or more sensors comprises automatically detecting, via a temperature sensor, the temperature of the fluid.

18. The computer-implemented method of claim 11, wherein receiving the infusion parameters comprises receiving the infusion parameters from a user input device.

19. The computer-implemented method of claim 18, wherein:
the infusion parameters further comprise a density of the fluid or a viscosity of the fluid; and
receiving the infusion parameters from the user input device comprises:
receiving a type of the fluid from the user input device; and
determining, based on the type of the fluid, the density of the fluid or the viscosity of the fluid.

20. The computer-implemented method of claim 11, wherein displaying the indication of the flow rate accuracy metric comprises displaying a percentage corresponding to the flow rate accuracy metric.
